(19) United States
(12) Patent Application Publication
(10) Pub. No.: US 2021/0363589 A1
(43) Pub. Date: Nov. 25, 2021

(54) IMMUNOTHERAPY USING MULTI-OMICS DATA TO EXTRACT MICROSATELLITE INSTABILITY-BASED NEOANTIGEN

(71) Applicant: Shenzhen NeoCura Biotechnology Corporation, Shenzhen (CN)

(72) Inventors: Ji WAN, Shenzhen (CN); Jian WANG, Shenzhen (CN); Yun-Wan XU, Shenzhen (CN); Youdong PAN, Shenzhen (CN); Yi WANG, Shenzhen (CN); Qi SONG, Shenzhen (CN)

(73) Assignee: Shenzhen NeoCura Biotechnology Corporation, Shenzhen (CN)

(21) Appl. No.: 16/992,113

(22) Filed: Aug. 13, 2020

(30) Foreign Application Priority Data
May 20, 2020 (CN) 202010427503.2

(51) Int. Cl. C12Q 1/6886; C12Q 1/6869; G16B 30/00

(52) U.S. Cl. CPC C12Q 1/6886; C12Q 1/6869; C12Q 2600/158; C12Q 2600/118; G16B 30/00

(57) ABSTRACT

A method is disclosed for integrating multi-omics data to extract a microsatellite instability (MSI)-based neoantigen for immunotherapy. The method includes the following steps: S1, integrating DNA and RNA sequencing data of a patient to detect the microsatellite instability (MSI) of the patient accurately; S2, translating open reading frames (ORFs) influenced by the detected MSI to acquire an MSI proteome; S3, mapping the MSI proteome against a normal human proteome to acquire a sample-specific proteome; and S4, acquiring a sample neoantigen. The new method reduces the rate of false positives in MSI detection, which is especially relevant for improving the efficacy of current clinical immunotherapy.

### CROSS REFERENCES TO THE RELATED APPLICATIONS

This application is based upon and claims priority to Chinese Patent Application No. 202010427503.2, filed on May 20, 2020, the entire contents of which are incorporated herein by reference.

### TECHNICAL FIELD

The present invention relates to the field of tumor immunotherapy, and in particular, to a method for integrating data of whole exome sequencing of DNA and RNA sequencing (RNA-seq) to extract a microsatellite instability (MSI)-related neoantigen for immunotherapy.

### BACKGROUND

The human immune system plays an important role in tumor therapy. In recent years, new immunotherapies based on the immune system have achieved breakthroughs in efficacy. These mechanisms achieve enhanced effects by recognizing the immune system and killing tumor cells by modifying T cells to activate the immune system or inhibit a system pathway. Among various types of immunotherapies, tumor neoantigen-based vaccines are well explored and developed. These vaccines are especially effective and have a wide application for various tumors, a short development cycle and few side effects.

The principle of the neoantigen vaccine is straightforward. Ten to twenty short peptides that may elicit immunogenicity are reinfused into the human body. This causes a proliferation of T cells that can recognize the short peptides. The peptides correspond in their structure to neoantigens on the surface of tumor cells. Thus, the T cells recognize and attach to the surface of the tumor and kill it, like an antibody kills bacteria.

Prediction of a neoantigen sequence requires high-throughput sequencing data of tissue DNAs and RNAs, along with bioinformatics and artificial intelligent (AI) technology. A general process is as follows: identifying DNA point mutations and small insertions/deletions, determining the expression of mutations with RNA sequencing (RNA-seq) data, and finally, determining whether a neoantigen elicits the immunogenicity by virtue of translation of open reading frames (ORFs) and integration of neoantigen-related multi-omics data. However, in a cell, pathways that generate neoantigens are not limited to DNA point mutations and insertions/deletions. Microsatellite instability (MSI)-induced repetitive DNA sequences are another common source for the generation of mutated polypeptides by tumor cells. However, in view of high false positive rate of MSI prediction based only on DNA, more diverse data and stricter filtering processes are required to ensure the clinical efficacy of neoantigens. Therefore, it is highly desirable to develop a high-precision method for predicting MSI-based neoantigens.

### SUMMARY

In view of the foregoing, the present invention addresses the likelihood that polypeptides generated by insertion/deletion of MSI in tumor tissues become neoantigens, and provides a bioinformatics method for acquiring tumor-specific neoantigens.

A first aspect of the present invention provides a method for integrating multi-omics data to extract MSI-based neoantigens for immunotherapy, including the following steps:

S1, integrating DNA and RNA sequencing data of a patient to detect the MSI locus of the patient;

S2, translating open reading frames (ORFs) associated with the detected MSI to acquire an MSI-related proteome;

S3, mapping against a normal human proteome to acquire a sample-specific proteome; and

S4, acquiring MSI-related neoantigen of the sample.

In some implementations, step S1 includes the following steps:

S101, acquiring candidate MSI from matched tumor/normal DNA sequencing data; and

S102, using RNA sequencing (RNA-seq) data of the patient to verify the expression of MSI-related DNA fragment acquired in step S101 to determine verified MSI.

In some implementations, step S101 includes the following steps:

S1011, pre-processing the Tumor/Normal sequencing data, including filtering of low-quality reads, alignment, and removal of repeated reads caused by PCR; and

S1012, with pre-processed Tumor/Normal bam as input, detecting tumor MSI of the patient by an MSI detection tool.

In some implementations, step S102 includes the following steps:

S1021, pre-processing the RNA-seq data, including filtering of low-quality reads, removal of adapters, and alignment; and

S1022, verifying detection results in step S101 one by one to acquire verified MSI mutations in conjunction with RNA alignment results obtained in step S1021.

In some implementations, step S2 includes the following steps:

S201, translating reading frames of MSI sequences after RNA expression validation to acquire MSI protein sequences, i.e., an MSI proteome; and

S202, fragmenting MSI proteins.

In some implementations, in step S3, all fragmented MSI peptide fragments are mapped against a normal human proteome and filtered to acquire brand-new candidate antigen peptides.

In some implementations, step S4 includes the following steps:

S401, using binary alignment map (bam) files obtained after DNA pre-processing in step S1 to genotype human leukocyte antigens (HLAs) of the sample;

S402, predicting the affinity of all brand-new candidate antigen peptides acquired in step S3 to sample-specific HLA molecules; and

S403, filtering sample neoantigens based on integrated peptide fragment information.

In some implementations, in step S403, candidate neoantigens are sorted and filtered to acquire a final tumor-specific MSI-based neoantigen by weighting different metrics.

In some implementations, specific metrics are selected from one or more of (i) the affinity of the peptide fragment to HLA, (ii) the expression of MSI-containing and normal transcripts in RNA-seq, (iii) the number of reads supporting MSI in tumor and normal samples in DNA sequencing and (iv) the physicochemical properties of the peptide fragments.

A second aspect of the present invention provides use of the method according to the first aspect in integrating multi-omics data to extract an MSI-based neoantigen for immunotherapy.

Compared with the prior art, the present invention has the following advantages:

1. In view of the source of the neoantigen, the method typically used is to acquire neoantigens by recognizing DNA point mutations and small insertions/deletions in somatic cells; tumor-specific neoantigens found by the method of the present invention are from the MSI and are widely present in a plurality of tumor types. Therefore, the present invention expands the screening range of neoantigens and enriches an “ammunition depot” of neoantigen-based immunotherapies.

2. In terms of the accuracy of MSI detection, the present invention integrates the genomic whole exome sequencing and RNA-seq data of a patient. By analyzing and integrating the data from these two sources, the false positive rate of the MSI detection is reduced to improve efficacy of neoantigen vaccines predicted by MSI, which is especially relevant for improving the efficacy of current clinical immunotherapy.

### BRIEF DESCRIPTION OF THE DRAWINGS

FIG. 1 is a flowchart of an exemplary method for integrating next-generation sequencing data of DNA and RNA to detect MSI-related neoantigens for immunotherapy of the present invention, where the text over the arrows and boxes represents processing steps and ribbon-shaped parts represent files.

### DETAILED DESCRIPTION OF THE EMBODIMENTS

The following paragraphs describe the present invention in detail through specific examples, but it should be noted that the embodiments are exemplary in nature. The present invention can also be implemented or applied through other embodiments. Based on different viewpoints and applications, various modifications or amendments can be made to the specification without departing from the spirit of the present invention.

Before further describing the specific examples of the present invention, it should be understood that the scope of protection of the present invention is not limited to the following specific examples; it should also be understood that the terms used herein are used for describing specific examples, rather than limiting the scope of protection of the present invention.

In order to enable those skilled in the art to better understand the present invention, the implementation of the present invention is described in detail below with reference to the drawing. The terms “first”, “second”, “again”, “then”, “next” used in specific examples herein are not intended to limit the order.

### Example 1

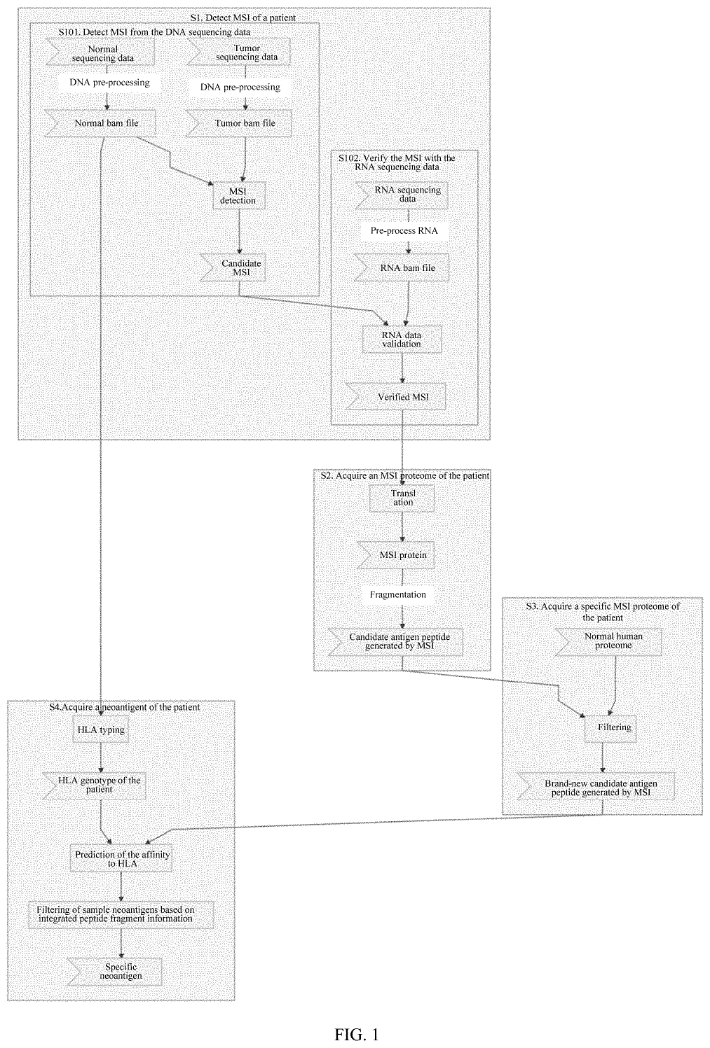

As shown in FIG. 1, part S1 is a flowchart of acquisition of genomic MSI of a tumor tissue based on whole exome sequencing implemented by a computer in the example of the present invention. The method includes the following steps executed by a computer:

S101, acquire possible MSI from tumor/normal matched DNA sequencing data.

S1011, pre-process the Tumor and Normal DNA sequencing data, respectively.

The primary objective of the preprocessing step is to remove PCR repeats to enable a more accurate result and generate a bam alignment file for subsequent analysis. Meanwhile, an optional step is to remove reads with a mean quality value of lower than 30 or 20 in sequencing.

Preferably, in the present invention, the acquisition of the genomic data of the sample is based on whole exome sequencing.

Preferably, in the present invention, the RNA-seq data of the sample is based on RNA-seq.

Preferably, repetitive sequences are removed from the sequencing data at a bam file level.

Preferably, bwa software is used to map sequenced fastq files to obtain a bam file, and then picard software is used to remove repetitive sequences from the bam file.

Command Lines and Parameters:

1. Mapping with bwa

bwa mem \

-R ‘@RG\tID: sample \tLB:library\tSM: sample’\
-t 20 \
-M bwa_index \
sample_1.DNA.fq.gz sample_2.DNA.fq.gz
where:
-R denotes a head file of an alignment result;
-t denotes the number of running threads;
-M denotes the index file used;
sample_1.DNA.fq.gz , sample_2.DNA.fq.gz is the original

sequencing data input.

2. Removing repetitive sequences by picard

java -jar picard.jar \

MarkDuplicate \
I=test.bam \
O=picard1.bam \
M=picard1.txt
where:
I denotes a bam file input;
O denotes a bam file output;
M denotes a statistical table of output results.

S1012, based on analysis methods provided by MSMuTect, detect tumor-specific MSI of samples from the pre-processed Tumor and Normal data.

In this step, according to the solution provided by MSMuTect, phobos is first used to extract sequences of microsatellite loci from a human reference genome and reads of microsatellite sequence present in the sequencing data, the data field is narrowed to increase the accuracy of results and reduce computation; then, tumor-specific MSI is detected by the kernel program of MSMuTect.

Preferably, in this step, it is necessary to filter the MSI that occurred outside the exon or use a detection tool for automatically filtering the MSI outside the exon (e.g., MSMuTect).

Operational Procedure:

1. Extraction of MSI regional sequences from a complete human reference genome and index building

(1) Extraction of MSI regional sequences from a complete human reference genome.

This step aims to splice upstream and downstream flanking bases at microsatellite loci in the human genome together as a reference sequence, excluding repetitive fragments per se. Specific operations are as follows:

a. MSI regions of the human genome are detected by phobos. The output format is required to be in the one-per-line format, and 5′-upstream (100 bp) and 3′-downstream sequences (100 bp) of microsatellite instable regions are included.

b. A script is written, and phobos results obtained in the previous step are converted into a file in fasta format.

Requirements:

Preserve records of MSI regions in exons;

splice upstream and downstream flanking regions in repeats together merely, where the sequence is composed of upstream flanking region and downstream flanking region, excluding repetitive fragments per se; and

classify different MSI regions into the corresponding fasta files according to types of repeat units.

Preferably, GRCh38 is selected as a human reference genome.

Preferably, the length of the flanking region is set as 100 bp for the upstream/downstream region.

Preferably, according to the solution provided by MSMuTect, only four typical repeat units are focused on: A, C, AC, and AG.

(2) Building of a sequence index of microsatellite regional reference sequences.

An index is built by using a bowtie2-build command for each reference sequence file corresponding to each repeat unit obtained in the previous step.

2. Extraction of reads with microsatellite sequence from sequencing data and mapping to a reference microsatellite sequence

The corresponding aln format alignment files are obtained after bam files of Tumor and Normal are processed as follows.

(1) converting bam files into the fastq format using bedtools;

(2) converting fastq format data into the fasta format

writing a script, and converting the pre-processed fastq sequencing data into the fasta format.

(3) extracting reads with microsatellite sequence by using phobos;

(4) converting results of phobos into the fasta format

where the specific operation of the step is similar to that of extraction of genomic microsatellite sequence, i.e., splicing upstream and downstream flanking regions of a microsatellite region together, with a requirement of the length of upstream/downstream sequence of at least 10 bp.

(5) mapping against the reference microsatellite sequence

using sequence alignment software bowtie2, mapping the sequences obtained in the previous step to the corresponding index generated in step (1) according to different repeat units.

3. Detection of microsatellite alterations.

Using MSMutect, tumor tissue-specific MSI alterations are detected by aln format alignment files of Tumor and Normal obtained in the previous step.

Command Lines and Parameters:

1. Converting the bam file format into the fastq file format

bedtools bamtofastq -i sample.bam -fq sample_R1.fastq -fq2
sample_R2.fastq
where:
-i denotes a whole exome sequencing alignment file;
-fq denotes reads at R1 end output in paired-end sequencing;
-fq2 denotes reads at R2 end output in paired-end sequencing.

2. Constructing a sequence index of MSI regions. A sample command of the step is:

bowtie2-build AC.fa dir/AC
where:
AC.fa indicates that repeat units obtained in the previous step are an

MSI sequence file of AC;

dir/C denotes a storage path to an index file.

3. Detecting MSI regions of human genome GRCh38 by phobos. A sample command of the step is:

phobos --minScore 5 -- minLength_b 5 --minUnitLen

1 --maxUnitLen 6--flanking 100 --outputFormat 3 GRCh38.fa
GRCh38.phobos

where:
--minScore denotes the minimum score of program output as 5;
--minLength_b denotes the repeat number of repeat units of the MSI
region as 5;
--minUnitLen denotes the minimum base number of a repeat unit as
1;
--maxUnitLen denotes the minimum base number of a repeat unit as
6;
--flanking denotes that an output result includes 5′-upstream (100 bp)

and 3′-downstream sequences (100 bp) of MSI regions are included;

--outputFormat denotes an output result format as 3, i.e., table format;
GRCh38.fa and GRCh38.phobos represent input and output files,
respectively.

### S102, Using RNA Sequencing (RNA-Seq) Data of the Patient to Verify the MSI Acquired in Step S101, to Acquire Verified MSI.

S1021, pre-process the RNA-seq data to obtain a BAM file.

The primary objective of the step is to obtain an aligned barn file, omit data quality control, and remove detailed descriptions of basic operations of adapters.

Preferably, STAR is used as alignment software.

Preferably, GRCh38 is selected as a human reference genome during alignment.

Command Lines and Parameters:

1. Mapping with STAR

STAR \

--runThreadN 20 \
--genomeDir star_index \
--readFilesIn sample_1.RNA.fq.gz sample_2.RNA.fq.gz \
--readFilesCommand zcat \
--outSAMtype BAM SortedByCoordinate \
--outSAMunmapped Within \
--outFilterMultimapNmax 1 \
--outFilterMismatchNmax 3 \
--chimSegmentMin 10 \
--chimOutType WithinBAM SoftClip \
--chimJunctionOverhangMin 10 \
--chimScoreMin 1 \
--chimScoreDropMax 30 \
--chimScoreJunctionNonGTAG 0 \
--chimScoreSeparation 1 \
--alignSJstitchMismatchNmax 5 −1 5 5 \
--chimSegmentReadGapMax 3
where:
--runThreadN denotes the number of threads to run;
--genomeDir denotes a path to an index file;
--readFilesIn denotes the original sequencing data read in;
--readFilesCommand denotes a command to read files;
--outSAMtype BAM SortedByCoordinate denotes the output format as BAM, while

sorting;

--outSAMunmapped Within denotes that unmapped reads are also output to a

destination file;

--outFilterMultimapNmax denotes the maximum number of loci the read isallowed

to map to;

--outFilterMismatchNmax denotes the maximum number of mismatches allowed;
--chimSegmentMin denotes output of a fusion transcript, and 10 represents the

number of the shortest mapped bases;

--chimOutType WithinBAM SoftClip denotes an output format of chimeric

alignment;

--chimJunctionOverhangMin denotes the minimum overhang for a chimeric

junction;

--chimScoreMin denotes the minimum total score of the chimeric segments;
--chimScoreDropMax denotes the maximum score drop among all chimeric

fragments;

--chimScoreJunctionNonGTAG denotes a penalty for a non-GT/AG chimeric

junction;

--chimScoreSeparation denotes the minimum difference between optimal and

suboptimal chimeric scores;

--alignSJstitchMismatchNmax denotes the maximum number of mismatches for

stitching of the splice junctions;

--chimSegmentReadGapMax denotes the maximum gap in the read sequence

between chimeric segments.

S1022, write a script to verify microsatellite alterations obtained in step S101 to acquire verified MSI.

Each detection result obtained in step S101 is verified according to the following steps:

1. First, construct a microsatellite allele sequence corresponding to the detection result.

According to a coordinate of the detection result, restore the microsatellite allele sequence of the patient: 10 bp upstream sequence+repeats (detected repeat units x number of repeats)+10 bp downstream sequence.

2. Then, verify whether microsatellite alteration sequences acquired from the DNA data are expressed in the RNA data.

According to the coordinate of the detection result, extract all reads mapped to the region from an RNA-seq alignment file;

Check whether the alteration sequences constructed in step 1 are present in these reads, and calculate the number of reads with these alteration sequences.

In FIG. 1, part S2 is a flowchart of acquisition of MSI proteome, including the following steps:

S201, translate reading frames of MSI sequences after RNA data validation to acquire MSI protein sequences, i.e., an MSI proteome.

First, make sure to enable verified MSI alteration regions to acquire all transcribed ORFs;

then, construct mutated transcripts and translate into mutant protein sequences.

S202, fragment MSI proteins.

A mutated peptide fragment is cleaved into small peptide fragments as peptide fragments of candidate neoantigens with tumor-specific MSI alterations.

A specific operational procedure of fragmentation is as follows:

A region able to produce an antigen peptide on the MSI protein is sliding-windowed in the presence of overlapping regions. If there is a fragment of 30 amino acids possibly generating a protein sequence of a neoantigen peptide, the length of peptide fragment will be set as 9, and peptide fragments selected will be: fragments 1 to 9, 2 to 10, 3 to 11, . . . , or 22 to 30.

Preferably, the default length of peptide fragment is set as 9 to 12 amino acids.

Preferably, it is necessary to determine whether a translational frameshift occurs when a reading frame is translated to an MSI locus; if the translational frameshift occurs, all protein sequences following MSI will be regarded as sources of potential neoantigen peptides; if the translational frameshift does not occur, only sequences in and around the MSI can produce neoantigen peptides.

In FIG. 1, part S3 is a flowchart of analysis of filtering antigens produced by MSI in a tumor of a patient by the present invention, including the following steps:

All fragmented MSI peptide fragments are mapped against a normal human proteome and filtered to acquire brand-new candidate antigen peptides.

Release 98 published by Ensembl is selected as the normal human proteome.

In FIG. 1, part S4 is a flowchart of analysis of filtering neoantigens produced by MSI in a tumor of a patient by the present invention, including the following steps:

S401, conduct molecular human leukocyte antigen (HLA) typing.

HLA genotypes are calculated using HLA genotyping software HLA-LA.

An example command is as follows:

HLA-LA.pl \

--BAM sample.bam \
--graph PRG_MHC_GRCh38_withIMGT \
--sampleID sample \
--maxThreads threads \
--workingDir out_dir \
--picard_sam2fastq_bin SamToFastq.jar
where:
--BAM denotes a bam file input;
--graph denotes a reference graph of population;
--sampleID denotes the unique identifier of the sample;
--maxThreads denotes the maximum number of threads;
--workingDir denotes an output path;
--picard_sam2fastq_bin denotes a tool for converting the bam file
into a fastq file.

S402, Predict the Affinity of Peptide Fragments.

Affinity prediction is conducted on MSI-specific peptide fragments from the patient's tumor generated in step S3 using netMHCpan-4.0 software and molecular HLA typing results.

An example command is as follows:

netMHCpan -BA -l 9 -a HLA_type -f filename -inptype 1 -xls -xlsfile
peptide.xls

where:
-BA denotes the conduct of affinity prediction;
-l denotes the length of peptide fragment;
-a denotes molecular HLA typing;
-f denotes an input file;
-inptype denotes the input file type, 0 = fasta file, and 1 = sequence
of the peptide fragment;
-xls denotes the output in the xls format;
-xlsfile denotes an output file name.

S403, Filter Sample Neoantigens Based on Integrated Peptide Fragment Information.

A script is written, peptide fragment information is integrated, and candidate neoantigens are sorted and filtered to acquire a final tumor-specific MSI-based neoantigen by weighting different metrics.

Specifically, first of all, make clear the source of every candidate peptide fragment, including gene names of ORFs and the corresponding transcript numbers, and annotate such information as (i) affinity of peptide fragment to HLA molecule, (ii) expression of expression of MSI-containing and normal transcripts in RNA-seq, (iii) number of reads supporting MSI in tumor and normal samples in DNA sequencing and (iv) specific position of a peptide fragment in a protein sequence.

At the filtering stage, candidate neoantigens are sorted and filtered to acquire a final tumor-specific MSI-based neoantigen by weighting different metrics. Specific metrics include (i) affinity of peptide fragment to HLA, (ii) expression of MSI-containing and normal transcripts in RNA-seq, (iii) number of reads supporting MSI in tumor and normal samples in DNA sequencing, and (iv) physicochemical properties of peptide fragments.

For the purposes of promoting an understanding of the principles of the invention, specific embodiments have been described. It should nevertheless be understood that the description is intended to be illustrative and not restrictive in character, and that no limitation of the scope of the invention is intended. Any alterations and further modifications in the described components, elements, processes or devices, and any further applications of the principles of the invention as described herein, are contemplated as would normally occur to one skilled in the art to which the invention pertains.

What is claimed is:

1. A method for integrating multi-omics data to extract a microsatellite instability (MSI)-based neoantigen for immunotherapy, comprising the following steps:
S1, integrating DNA sequencing (DNA-seq) data and RNA sequencing (RNA-seq) data of a sample from a patient to detect tumor-specific MSI of the patient;
S2, translating open reading frames (ORFs) associated with the tumor-specific MSI to acquire an MSI proteome;
S3, mapping the MSI proteome against a normal human proteome to acquire a sample-specific proteome; and
S4, acquiring a sample neoantigen.

2. The method according to claim 1, wherein step S1 comprises the following steps:
S101, acquiring candidate tumor MSI from Tumor/Normal matched DNA sequencing data; and
S102, using the RNA sequencing (RNA-seq) data of the patient to verify the candidate tumor-specific MSI acquired in step S101, to acquire verified tumor-specific MSI.

3. The method according to claim 1, wherein step S101 comprises the following steps:
S1011, pre-processing the Tumor/Normal matched DNA sequencing data, comprising filtering of low-quality reads, alignment, and removal of PCR duplicates; and
S1012, with a pre-processed Tumor/Normal bam as input, detecting the candidate tumor-specific MSI of the patient by an MSI detection tool.

4. The method according to claim 1, wherein step S102 comprises the following steps:
S1021, pre-processing the RNA-seq data, comprising filtering of low-quality reads, removal of adapters, and alignment; and
S1022, verifying detection results in step S101 one by one to acquire the verified tumor-specific MSI in conjunction with RNA alignment results obtained in step S1021.

5. The method according to claim 1, wherein step S2 comprises the following steps:
S201, translating open reading frames of the tumor-specific MSI sequences after RNA data validation to acquire MSI protein sequences, i.e., an MSI proteome; and
S202, fragmenting the MSI protein sequences.

6. The method according to claim 1, wherein, in step S3, all peptide fragments fragmented from the MSI proteome are mapped against a normal human proteome and filtered to acquire brand-new candidate antigen peptides.

7. The method according to claim 1, wherein step S4 comprises the following steps:
S401, using bam files obtained after DNA pre-processing in step S1 to genotype human leukocyte antigens (HLAs) of the sample;
S402, predicting affinity scores of all brand-new candidate antigen peptides acquired in step S3 to sample-specific HLA molecules; and
S403, filtering sample neoantigens based on integrated peptide fragment information.

8. The method according to claim 7, wherein, in step S403, the sample neoantigens are sorted and filtered to acquire a final tumor-specific MSI-based neoantigen using different metrics and corresponding weights.

9. The method according to claim 8, wherein the different metrics are specifically selected from one or more of a group consisting of affinity of peptide fragment to HLA, expression of MSI-containing and normal transcripts in RNA-seq, number of reads supporting MSI in tumor and normal samples in DNA sequencing, and physicochemical properties of peptide fragments.

10. An application of the method according to claim 1 in integrating multi-omics data to extract an MSI-based neoantigen for immunotherapy.

11. The method according to claim 3, wherein step S1 comprises the following steps:
S101, acquiring the candidate tumor MSI from the Tumor/Normal matched DNA sequencing data; and
S102, using the RNA sequencing (RNA-seq) data of the patient to verify the candidate tumor-specific MSI acquired in step S101, to acquire the verified tumor-specific MSI.

12. The method according to claim 4, wherein step S1 comprises the following steps:
S101, acquiring the candidate tumor MSI from the Tumor/Normal matched DNA sequencing data; and
S102, using the RNA sequencing (RNA-seq) data of the patient to verify the candidate tumor-specific MSI acquired in step S101, to acquire the verified tumor-specific MSI.

13. The method according to claim 4, wherein step S101 comprises the following steps:
S1011, pre-processing the Tumor/Normal matched DNA sequencing data, comprising filtering of the low-quality reads, alignment, and removal of the PCR duplicates; and
S1012, with the pre-processed Tumor/Normal bam as input, detecting the candidate tumor-specific MSI of the patient by the MSI detection tool.

14. The method according to claim 5, wherein step S1 comprises the following steps:
S101, acquiring the candidate tumor MSI from the Tumor/Normal matched DNA sequencing data; and
S102, using the RNA sequencing (RNA-seq) data of the patient to verify the candidate tumor-specific MSI acquired in step S101, to acquire the verified tumor-specific MSI.

15. The method according to claim 5, wherein step S101 comprises the following steps:
S1011, pre-processing the Tumor/Normal matched DNA sequencing data, comprising filtering of the low-quality reads, alignment, and removal of the PCR duplicates; and
S1012, with the pre-processed Tumor/Normal bam as input, detecting the candidate tumor-specific MSI of the patient by the MSI detection tool.

16. The method according to claim 5, wherein step S102 comprises the following steps:
S1021, pre-processing the RNA-seq data, comprising filtering of the low-quality reads, removal of the adapters, and alignment; and
S1022, verifying the detection results in step S101 one by one to acquire the verified tumor-specific MSI in conjunction with the RNA alignment results obtained in step S1021.

17. The method according to claim 6, wherein step S1 comprises the following steps:
S101, acquiring the candidate tumor MSI from the Tumor/Normal matched DNA sequencing data; and
S102, using the RNA sequencing (RNA-seq) data of the patient to verify the candidate tumor-specific MSI acquired in step S101, to acquire the verified tumor-specific MSI.

18. The method according to claim 6, wherein step S101 comprises the following steps:
S1011, pre-processing the Tumor/Normal matched DNA sequencing data, comprising filtering of the low-quality reads, alignment, and removal of the PCR duplicates; and
S1012, with the pre-processed Tumor/Normal bam as input, detecting the candidate tumor-specific MSI of the patient by the MSI detection tool.

19. The method according to claim 6, wherein step S102 comprises the following steps:
S1021, pre-processing the RNA-seq data, comprising filtering of the low-quality reads, removal of the adapters, and alignment; and
S1022, verifying the detection results in step S101 one by one to acquire the verified tumor-specific MSI in conjunction with the RNA alignment results obtained in step S1021.

20. The method according to claim 6, wherein step S2 comprises the following steps:
S201, translating the open reading frames of the tumor-specific MSI sequences after RNA data validation to acquire the MSI protein sequences, i.e., the MSI proteome; and
S202, fragmenting the MSI protein sequences.
